(19) United States
(12) Patent Application Publication
(10) Pub. No.: US 2018/0036049 A1
(43) Pub. Date: Feb. 8, 2018

(54) BONE PLATE AND BONE PLATE SYSTEM

(71) Applicant: KOBAYASHI PRECISION INDUSTRY CO., LTD., Sagamihara-shi, Kanagawa (JP)

(72) Inventor: Masazumi KOBAYASHI, Machida-shi, Tokyo (JP)

(21) Appl. No.: 15/311,728

(22) PCT Filed: Feb. 8, 2016
(86) PCT No.: PCT/JP2016/053682

§ 371 Date: Nov. 16, 2016

(51) Int. Cl. A61B 17/80; A61B 17/17

(52) U.S. Cl. CPC A61B 17/8052; A61B 17/1728; A61B 17/8033

(57) ABSTRACT

A screw can be fastened to a bone at a desired angle. A bone plate includes: an upper surface on a side opposite to a bone; a lower surface on a side closer to the bone; and at least one first hole connecting the upper surface and the lower surface and being configured to allow insertion of a screw. The first hole has an upper inner peripheral surface on an upper surface side and a lower inner peripheral surface on a lower surface side. The upper inner peripheral surface has an inner diameter decreasing toward the lower surface. The lower inner peripheral surface has an inner diameter increasing toward the lower surface. The lower inner peripheral surface includes at least one first region inclined at a first angle with respect to a center axis of the first hole, and at least one second region inclined at a second angle with respect to the center axis of the first hole.

### TECHNICAL FIELD

The present invention relates to a bone plate and a bone plate system.

### BACKGROUND ART

There has been known a bone plate to be fixed to a bone through use of a screw having a male thread formed on a head portion. For example, a bone plate having a fixation hole is disclosed in Patent Literature 1. The fixation hole has a female thread formed so as to be engaged with the male thread formed on the head portion of the screw.

### CITATION LIST

### Patent Literature

Patent Literature 1: Japanese Patent Translation Publication No. 2015-511859

### SUMMARY OF INVENTION

### Technical Problem

The female thread is formed in advance on an inner surface of the fixation hole of the bone plate disclosed in Patent Literature 1. Thus, an axial direction of the screw driven into the fixation hole is determined by a center axis direction of the fixation hole. However, an angle of the screw to the bone is required to be appropriately determined in consideration of a surface shape of the bone, strength of the bone, and the like. With the bone plate disclosed in Patent Literature 1, it is difficult to fasten the screw to the bone at a desired angle unless a surgeon deforms the bone plate or reprocesses the fixation hole.

It is an object of the present invention to provide a bone plate and a bone plate system that enable a screw to be fastened to a bone at a desired angle.

### Solution to Problem

In order to solve the above-mentioned problem, according to one embodiment of the present invention, there is provided a bone plate, including: an upper surface on a side opposite to a bone; a lower surface on a side closer to the bone; and at least one first hole connecting the upper surface and the lower surface and being configured to allow insertion of a screw. The first hole has an upper inner peripheral surface on an upper surface side and a lower inner peripheral surface on a lower surface side. The upper inner peripheral surface has an inner diameter decreasing toward the lower surface, and the lower inner peripheral surface has an inner diameter increasing toward the lower surface. The lower inner peripheral surface includes at least one first region inclined at a first angle with respect to a center axis of the first hole and at least one second region inclined at a second angle with respect to the center axis of the first hole.

In the above-mentioned bone plate, the screw may include a head portion and a shaft portion, each having a male thread formed thereon. The lower inner peripheral surface may have no female thread formed in advance, and the female thread may be formed through rotation of the head portion after the screw is inserted into the first hole.

In the above-mentioned bone plate, the lower inner peripheral surface may include one set of the first regions facing each other across the center axis of the first hole and one set of the second regions facing each other across the center axis of the first hole.

In the above-mentioned bone plate, the one set of first regions may be arranged so as to face each other in a longitudinal direction of the bone plate, and the one set of second regions may be arranged so as to face each other in a direction intersecting with the longitudinal direction of the bone plate.

In the above-mentioned bone plate, the first angle maybe larger than the second angle.

In the above-mentioned bone plate, a plurality of the first holes may be arranged at intervals in a longitudinal direction of the bone plate, and at least one of the plurality of first holes may include the one set of first regions arranged so as to face each other in the longitudinal direction of the bone plate and the one set of second regions arranged so as to face each other in the direction intersecting with the longitudinal direction of the bone plate.

The above-mentioned bone plate may further include at least one second hole connecting the upper surface and the lower surface and being configured to allow insertion of the screw. The second hole may have an upper inner peripheral surface on the upper surface side and a lower inner peripheral surface on the lower surface side. The upper inner peripheral surface may have an inner diameter decreasing toward the lower surface, and the lower inner peripheral surface may have an inner diameter in a predetermined direction increasing toward the lower surface. The lower inner peripheral surface may include at least one first region inclined at a first angle with respect to a center axis of the second hole and at least one second region in parallel to the center axis of the second hole.

In order to solve the above-mentioned problem, according to another embodiment of the present invention, there is provided a bone plate system including the above-mentioned bone plate and the screw. The screw includes a head portion and a shaft portion, each having a male thread formed thereon. The lower inner peripheral surface has no female thread formed in advance, and the female thread is formed through rotation of the head portion after the screw is inserted into the first hole.

### Advantageous Effects of Invention

According to the present invention, the screw can be fastened to a bone at a desired angle.

### BRIEF DESCRIPTION OF DRAWINGS

FIG. 1 is a perspective view of a bone plate system according to one embodiment of the present invention.

FIG. 2 is a side view of the bone plate system in a longitudinal direction.

FIG. 3A and FIG. 3B are side views of the bone plate system in a width direction. FIG. 3A is a view for illustrating screws in a center, and FIG. 3B is a view for illustrating screws at an end portion.

FIG. 4 is a side view of the screw.

FIG. 5 is a perspective view of an upper surface side of a bone plate.

FIG. 6 is a perspective view of a lower surface side of the bone plate.

FIG. 7 is a plan view of the bone plate.

FIG. 8 is a bottom view of the bone plate.

FIG. 9 is a side view of the bone plate in the longitudinal direction.

FIG. 10 is a side view of the bone plate in the width direction.

FIG. 11 is a partially enlarged view of the bone plate (portion F of FIG. 5).

FIG. 12 is a partially enlarged view of the bone plate (portion G of FIG. 6).

FIG. 13 is a sectional view of the bone plate (taken along the line A-A of FIG. 7) in the longitudinal direction.

FIG. 14A, FIG. 14B, and FIG. 14C are enlarged partial sectional views of the bone plate (portion E of FIG. 13). FIG. 14A is a view for illustrating a state before a screw is inserted. FIG. 14B is a view for illustrating a state in which the screw is driven straight. FIG. 14C is a view for illustrating a state in which the screw is driven obliquely.

FIG. 15A, FIG. 15B, and FIG. 15C are enlarged partial sectional views of the bone plate (portion C-C of FIG. 7). FIG. 15A is a view for illustrating a state before the screw is inserted. FIG. 15B is a view for illustrating a state in which the screw is driven straight. FIG. 15C is a view for illustrating a state in which the screw is driven obliquely.

FIG. 16 is an enlarged partial sectional view of the bone plate (portion D of FIG. 13).

FIG. 17 is an enlarged sectional view of the bone plate (taken along the line B-B of FIG. 7).

FIG. 18A, FIG. 18B, FIG. 18C, and FIG. 18D are side views for illustrating a schematic configuration of a jig. FIG. 18A is a view for illustrating an internal configuration of the jig. FIG. 18B is a view for illustrating a state in which the jig is placed upright. FIG. 18C is a view for illustrating a state in which the jig is placed obliquely. FIG. 18D is another view for illustrating a state in which the jig is placed obliquely.

### DESCRIPTION OF EMBODIMENTS

Now, one embodiment of the present invention is described with reference to the drawings.

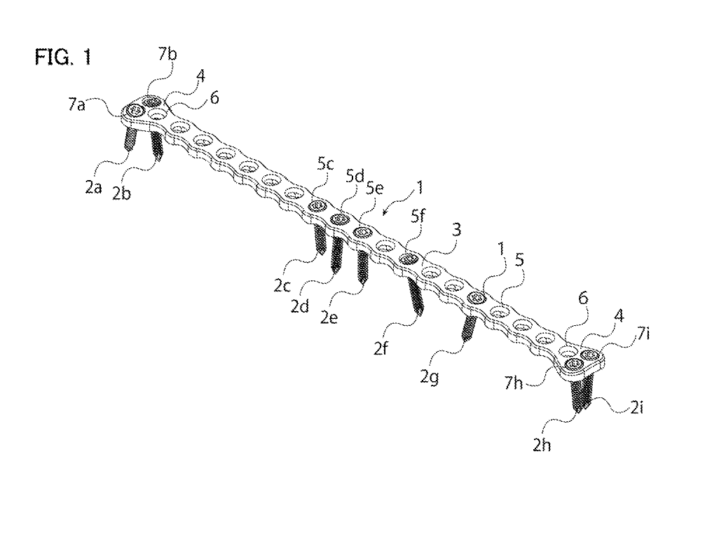

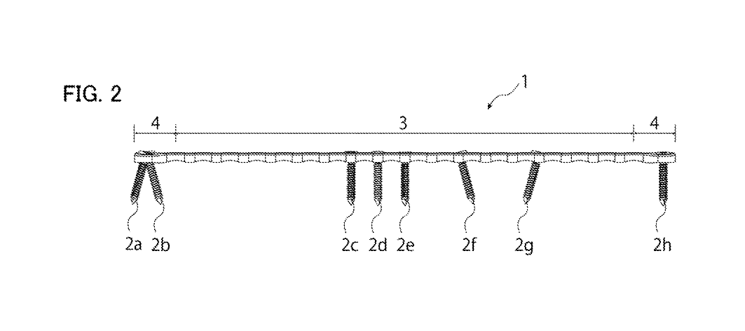

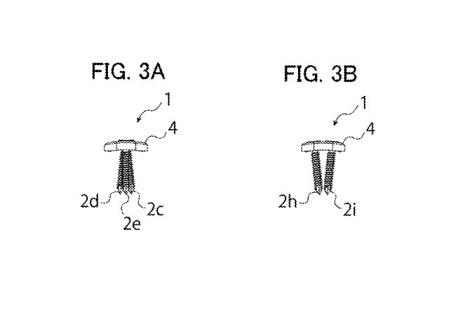

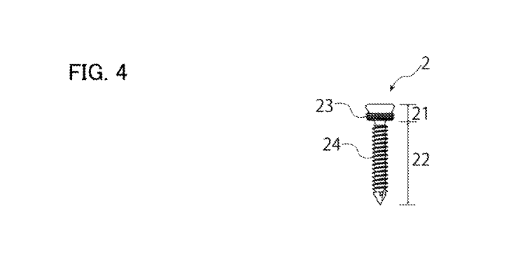

FIG. 1 is a perspective view of a bone plate system according to one embodiment of the present invention. FIG. 2 is a side view of the bone plate system in a longitudinal direction. FIG. 3A and FIG. 3B are side views of the bone plate system in a width direction. FIG. 3A is a view for illustrating screws in a center, and FIG. 3B is a view for illustrating screws at an end portion. FIG. 1 to FIG. 3D are views for illustrating an example in which a plurality of screws 2 (2a to 2i) are fastened to a bone plate 1. FIG. 3A is a view of the three screws 2c, 2d, and 2e as viewed from the screw 2e side. FIG. 3B is a view of the two screws 2h and 2i. FIG. 4 is a side view of the screw 2. In FIG. 4, the screw 2 is illustrated in an enlarged manner.

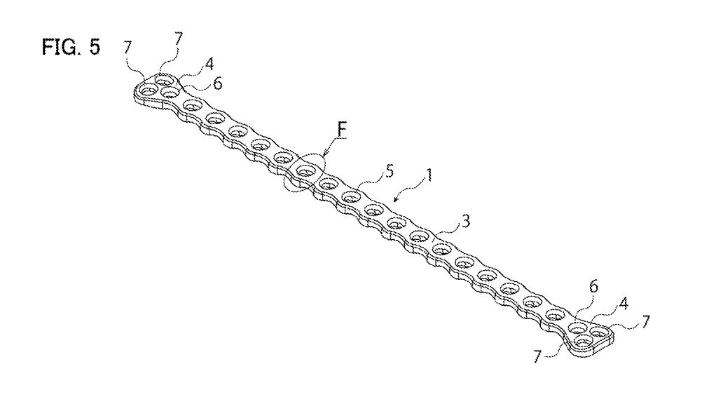

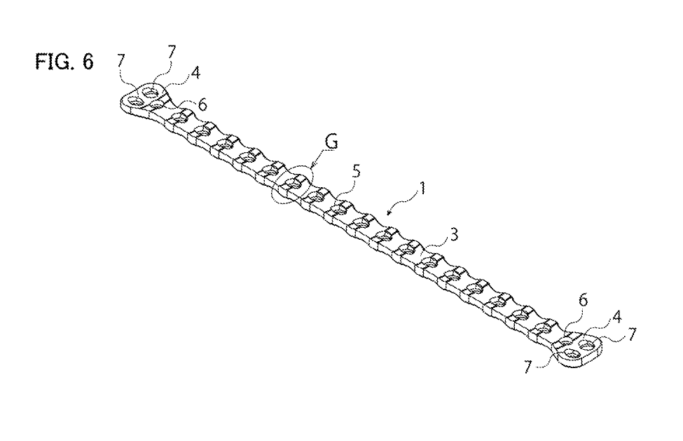

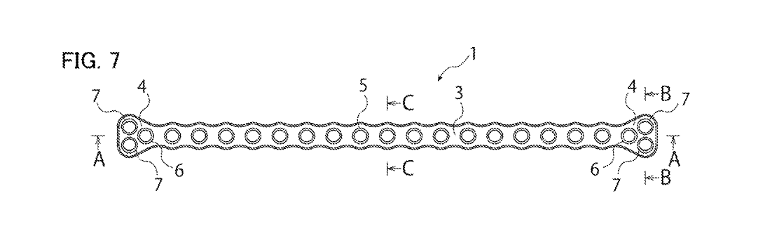

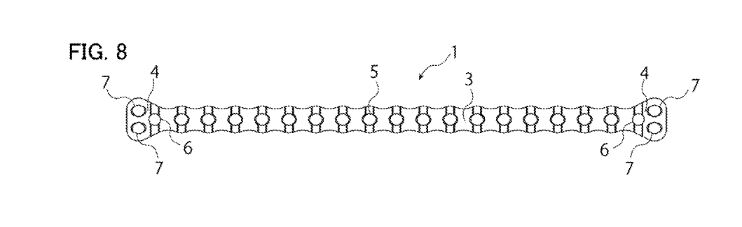

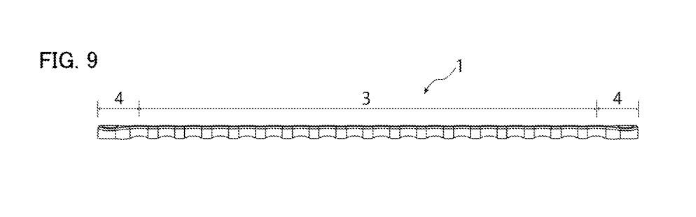

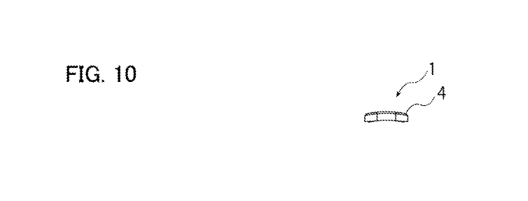

FIG. 5 is a perspective view of an upper surface side of the bone plate. FIG. 6 is a perspective view of a lower surface side of the bone plate. FIG. 7 is a plan view of the bone plate. FIG. 8 is a bottom view of the bone plate. FIG. 9 is a side view of the bone plate in the longitudinal direction. FIG. 10 is a side view of the bone plate in the width direction. Both side surfaces of the bone plate in the longitudinal direction have the same shape. Further, both side surfaces of the bone plate in the width direction have the same shape.

The bone plate system includes the bone plate 1 and at least one screw 2. The bone plate 1 is formed into an elongated flat plate-like shape as a whole. Further, the bone plate 1 is formed so as to curve in an arc-like shape as viewed in the longitudinal direction so that a lower surface of the bone plate 1 (surface on a lower side in FIG. 2) is formed along a surface shape of a bone. As one example, the bone plate system is used to join and reinforce bones of small animals including dogs and cats. For example, a dimension of the bone plate 1 in the longitudinal direction can be set to about 100 mm. A dimension of the bone plate 1 in the width direction can be set to about 10 mm. A dimension of the bone plate 1 in a thickness direction can be set to about 2.5 mm. A material of the bone plate 1 is, for example, metal such as a titanium alloy, a resin, or the like.

The bone plate 1 includes a center portion 3 and end portions 4 at both ends of the center portion 3. A maximum width dimension of each of the end portions 4 is larger than a maximum width dimension of the center portion 3.

In the center portion 3 of the bone plate 1, a plurality of (seventeen in FIG. 1) holes 5 are formed in line at equal intervals in the longitudinal direction. The width dimension of the center portion 3 repeatedly changes so as to be maximum at a center of each of the holes 5 in the longitudinal direction and minimum at each of intermediate positions between the adjacent holes 5. Further, the width dimension of the center portion 3 is minimum at a position at which the center portion 3 is connected to each of the end portions 4 in the longitudinal direction. A thickness dimension of the center portion 3 repeatedly changes so as to be maximum at the center of each of the holes 5 in the longitudinal direction and minimum at each of the intermediate positions between the adjacent holes 5. The shape of the lower surface of the bone plate 1 can reduce a contact area with a surface of the bone as compared to a case where the shape of the lower surface of the bone plate is flat.

In each of the end portions 4 of the bone plate 1, a hole 6 is formed on a side closer to the center portion 3. Further, in each of the end portions 4, two holes 7 are formed on a side farther from the center portion 3 with an interval in the width direction. The width dimension of each of the end portions 4 is increased so as to be minimum at a position at which the end portion 4 is connected to the center portion 3 and maximum at a center of the two holes 7 in the longitudinal direction.

Each of the screws 2 includes a head portion 21 and a shaft portion 22. A male thread 23 is formed on a lower portion of the head portion 21. A male thread 24 is formed on the shaft portion 22. The screw 2 can also be called a “locking screw”. An outer diameter of the head portion 21 is larger than an outer diameter of the shaft portion 22. The outer diameter of the shaft portion 22 is smaller than each of inner diameters (minimum inner diameters) of the holes 5 to 7. The outer diameter of the head portion 21 is larger than each of inner diameters (minimum inner diameters) of the holes 5 to 7. Through rotation of the screw 2, an inner wall of a corresponding one of the holes 5 to 7 is scraped by the male thread 23 of the head portion 21 (corresponding one of the holes 5 to 7 is subjected to tapping). As a result, the head portion 21 of the screw 2 is fixed to the corresponding one of the holes 5 to 7.

The holes 5 connect an upper surface (surface on an upper side in FIG. 2) of the bone plate 1 and a lower surface (surface on a lower side in FIG. 2) of the bone plate 1 to each other. A center axis of each of the holes 5 is approximately perpendicular to the upper surface and the lower surface of the bone plate 1. Each of the holes 5 has an approximately cylindrical shape as a whole. A female thread is not formed in advance on an inner surface of each of the holes 5 (the inner surface of each of the holes 5 is not subjected to tapping). The female thread is cut by the male thread 23 of the head portion 21 of the screw 2. The screws 2 can be driven into the holes 5 at various angles. For example, as illustrated in FIG. 1 to FIG. 3B, the screw 2e is driven into a hole 5e so that an axis of the screw 2e matches a center axis of the hole 5e. The screw 2d is driven into a hole 5d so that an axis of the screw 2d is inclined with respect to a center axis of the hole 5e in the width direction of the bone plate 1. The screw 2f is driven into a hole 5f so that an axis of the screw 2f is inclined with respect to a center axis of the hole 5f in the longitudinal direction of the bone plate 1. A structure of each of the holes 5 for achieving the above-mentioned function is described later in detail.

The holes 6 connect the upper surface of the bone plate 1 and the lower surface of the bone plate 1 to each other. Each of the holes 6 has an approximately cylindrical shape as a whole. A center axis of each of the holes 6 is approximately perpendicular to the upper surface and the lower surface of the bone plate 1. A female thread is not formed in advance on an inner surface of each of the holes 6 (the inner surface of each of the holes 6 is not subjected to tapping). The female thread is cut by the male thread 23 of the head portion 21 of each of the screws 2. The screws 2 can be driven into the holes 6 at a predetermined angle. For example, the screw 2 is driven into a corresponding one of the holes 6 so that the axis of the screws 2 matches a center axis of the corresponding one of the holes 6. A structure of each of the holes 6 for achieving the above-mentioned function is described later in detail.

The holes 7 connect the upper surface of the bone plate 1 and the lower surface of the bone plate 1 to each other. A center axis of each of the holes 7 is approximately perpendicular to the upper surface and the lower surface of the bone plate 1. Each of the holes 7 has an approximately cylindrical shape as a whole. A female thread is not formed in advance on an inner surface of each of the holes 7 (the inner surface of each of the holes 7 is not subjected to tapping). The female thread is cut by the male thread 23 of the head portion 21 of each of the screws 2. The screws 2 can be driven into the holes 7 at various angles. For example, as illustrated in FIG. 1 to FIG. 3B, the screw 2h is driven into a hole 7h so that an axis of the screw 2h matches a center axis of the hole 7h. The screw 2a is driven into a hole 7a so that an axis of the screw 2a is inclined with respect to a center axis of the hole 7a in the longitudinal direction of the bone plate 1. A structure of each of the holes 7 for achieving the above-mentioned function is described later in detail.

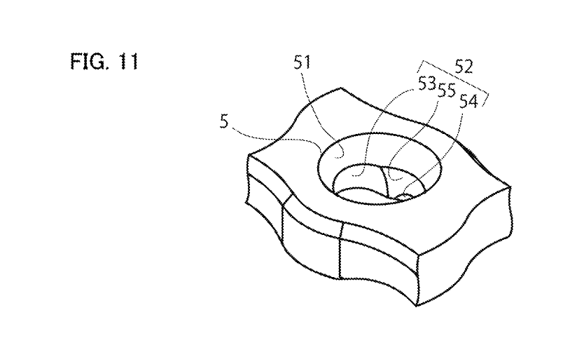

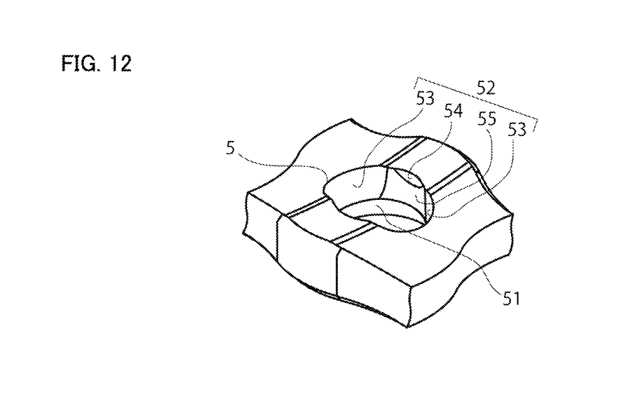

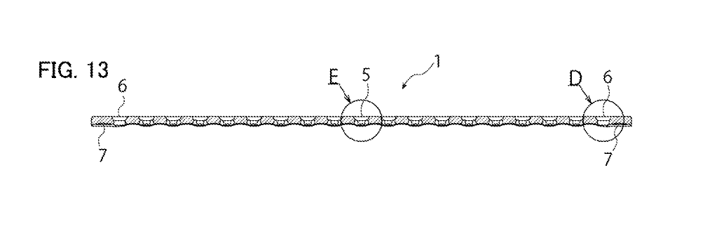

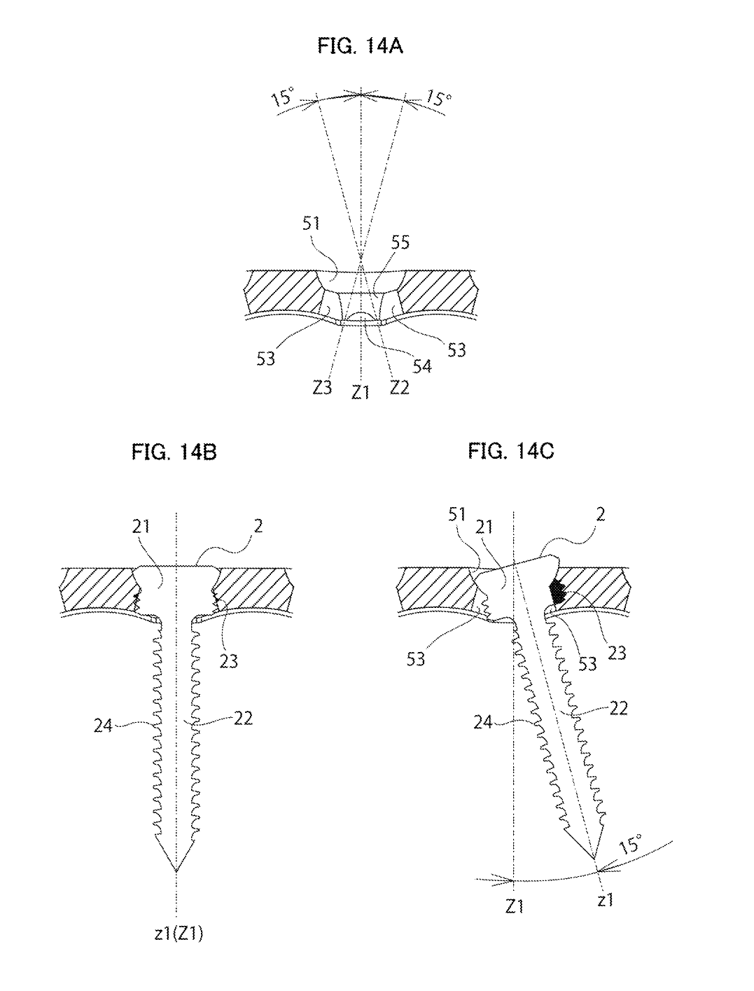

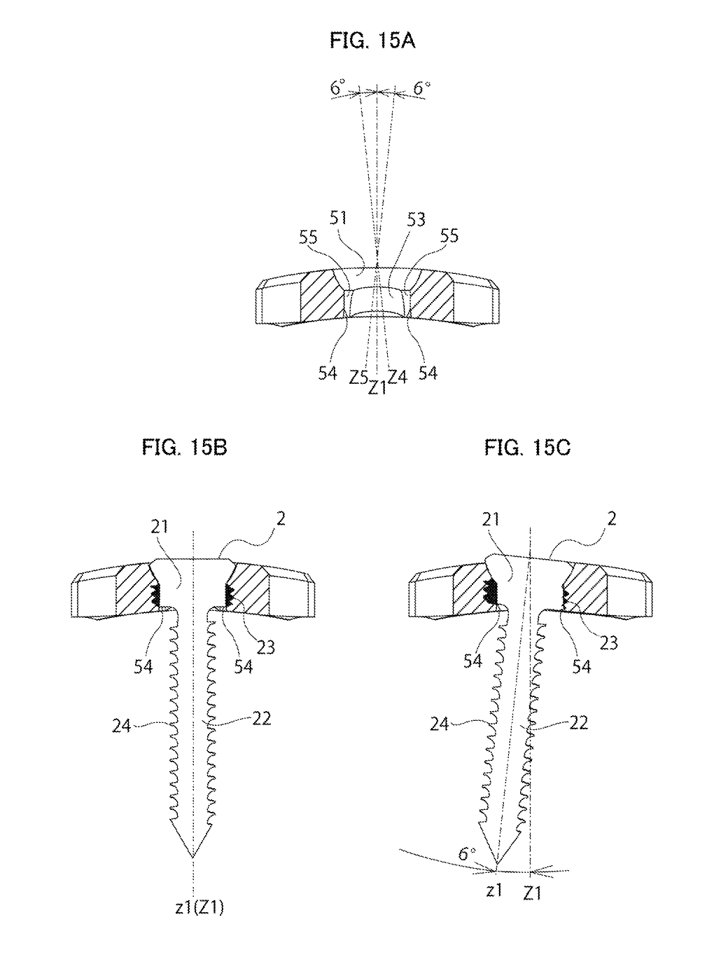

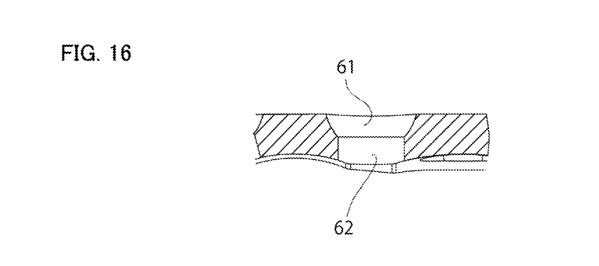

FIG. 11 is a partially enlarged view of the bone plate (portion F of FIG. 5). FIG. 12 is a partially enlarged view of the bone plate (portion G of FIG. 6). FIG. 13 is a sectional view of the bone plate (taken along the line A-A of FIG. 7) in the longitudinal direction. FIG. 14A, FIG. 14B, and FIG. 14C are enlarged partial sectional views of the bone plate (portion E of FIG. 13). FIG. 14A is a view for illustrating a state before a screw is inserted. FIG. 14B is a view for illustrating a state in which the screw is driven straight. FIG. 14C is a view for illustrating a state in which the screw is driven obliquely. FIG. 15A, FIG. 15B, and FIG. 15C are enlarged partial sectional views of the bone plate (portion C-C of FIG. 7). FIG. 15A is a view for illustrating a state before a screw is inserted. FIG. 15B is a view for illustrating a state in which the screw is driven straight. FIG. 15C is a view for illustrating a state in which the screw is driven obliquely. FIG. 16 is an enlarged partial sectional view of the bone plate (portion D of FIG. 13). In FIG. 14A to FIG. 15C, a black area represents a cross section of a region where the screw 2 and the hole 5 are engaged with each other.

As illustrated in FIG. 11 and FIG. 12, each of the holes 5 has an upper inner peripheral surface 51 on an upper surface side of the bone plate 1 and a lower inner peripheral surface 52 on a lower surface side of the bone plate 1. The upper inner peripheral surface 51 is formed along a shape of the upper portion of the head portion 21 of the screw 2. The upper inner peripheral surface 51 is formed of a conical surface so that an inner diameter thereof decreases toward the lower surface. The lower inner peripheral surface 52 includes a plurality of conical surfaces so that an inner diameter thereof increases toward the lower surface as a whole. The lower inner peripheral surface 52 includes first regions 53 facing each other, each being inclined at a first angle (larger than 0 degrees) with respect to the center axis of the hole 5. The lower inner peripheral surface 52 also includes second regions 54 facing each other, each being inclined at a second angle (larger than 0 degrees) with respect to the center axis of the hole 5. One set of the first regions 53 facing each other across the center axis of the hole 5 is formed on the lower inner peripheral surface 52 so as to face each other in the longitudinal direction of the bone plate 1. Further, one set of the second regions 54 facing each other across the center axis of the hole 5 is formed on the lower inner peripheral surface 52 so as to face each other in the width direction of the bone plate 1. On an upper side of the second regions 54, third regions 55 facing each other in parallel to the center axis of the hole 5 are formed so as to face each other in the width direction of the bone plate 1.

The screw 2 can be driven into the hole 5 described above so that the axis of the screw 2 matches the center axis of the hole 5. Further, the screw 2 can be driven into the hole 5 so that the axis of the screw 2 is inclined in the longitudinal direction or the width direction of the bone plate 1 with respect to the center axis of the hole 5.

As illustrated in FIG. 14A, the first regions 53 face each other, each being inclined at the first angle (15 degrees in FIG. 14A) with respect to a center axis Z1 of the hole 5. FIG. 14B is an illustration of a case where the screw 2 is driven into the hole 5 so that an axis z1 of the screw 2 matches the center axis Z1. In this case, an entire periphery of the male thread 23 of the head portion 21 is engaged with the female thread formed on the first regions 53 and the second regions 54. An outer peripheral surface of the upper portion of the head portion 21 is held in contact with the upper inner peripheral surface 51 to receive a reactive force generated by tightening the screw 2. FIG. 14C is an illustration of a case where the screw 2 is driven into the hole 5 so that the axis z1 of the screw 2 is inclined at the first angle (15 degrees in FIG. 14C) in the longitudinal direction of the bone plate 1 (direction in which the first regions 53 face each other) with respect to the center axis Z1. In this case, the periphery of the male thread 23 of the head portion 21 is at least partially engaged with the female thread formed on the first regions 53 and the second regions 54. In FIG. 14C, the male thread 23 of the head portion 21 is engaged with the first region 53 corresponding to the direction of inclination and the one set of second regions 54. The outer peripheral surface of the upper portion of the head portion 21 is held in contact with the upper inner peripheral surface 51 to receive a reactive force generated by tightening the screw 2.

As illustrated in FIG. 15A, the second regions 54 face each other, each being inclined at the second angle (6 degrees in FIG. 15A) with respect to the center axis Z1 of the hole 5. FIG. 15B is an illustration of a case where the screw 2 is fastened into the hole 5 so that the axis z1 of the screw 2 matches the center axis Z1. In this case, the entire periphery of the male thread 23 of the head portion 21 is engaged with the female thread formed on the first regions 53 and the second regions 54. The outer peripheral surface of the upper portion of the head portion 21 is held in contact with the upper inner peripheral surface 51 to receive a reactive force generated by tightening the screw 2. FIG. 15C is an illustration of a case where the screw 2 is driven into the hole 5 so that the axis z1 of the screw 2 is inclined at the second angle (6 degrees in FIG. 15C) in the width direction of the bone plate 1 (direction in which the second regions 54 face each other) with respect to the center axis Z1. In this case, the periphery of the male thread 23 of the head portion 21 is at least partially engaged with the female thread formed on the first regions 53 and the second regions 54. In FIG. 15C, the entire periphery of the male thread 23 of the head portion 21 is engaged with the female thread formed on the first regions 53 and the second regions 54. The outer peripheral surface of the upper portion of the head portion 21 is held in contact with the upper peripheral surface 51 to receive a reactive force generated by tightening the screw 2.

The two first regions 53 that face each other can be formed in the following manner. Specifically, a hole is formed along the center axis Z1 through use of a drill, and the upper inner peripheral surface 51 is formed by machining through use of a ball-end mill along the center axis Z1. After that, the first regions 53 are formed by machining through use of the ball-end mill along an axis Z2 and an axis Z3 respectively inclined at the first angle (15 degrees in FIG. 14A) in both directions in the longitudinal direction with respect to the center axis Z1.

The two second regions 54 that face each other can be formed in the following manner. Specifically, the hole is formed along the center axis Z1 through use of a drill, and the upper inner peripheral surface 51 is formed by machining through use of a ball-end mill along the center axis Z1. After that, the second regions 54 are formed by machining through use of the ball-end mill along an axis Z4 and an axis Z5 respectively inclined at the second angle (6 degrees in FIG. 15A) in both directions in the width direction with respect to the center axis Z1. The third regions 55 are formed when the hole is formed along the center axis Z1.

As illustrated in FIG. 16, the hole 6 includes an upper inner peripheral surface 61 on the upper surface side of the bone plate 1 and a lower inner peripheral surface 62 on the lower surface side of the bone plate 1. The upper inner peripheral surface 61 is formed along the shape of the upper portion of the head portion 21 of the screw 2. The upper inner peripheral surface 61 is formed of a conical surface so that an inner diameter thereof decreases toward the lower surface. An inner diameter of the lower inner peripheral surface 62 is formed uniformly. Specifically, the lower inner peripheral surface 62 is entirely parallel to a center axis of the hole 6.

The screw 2 can be driven into the hole 6 described above so that the axis of the screw 2 matches the center axis of the hole 6.

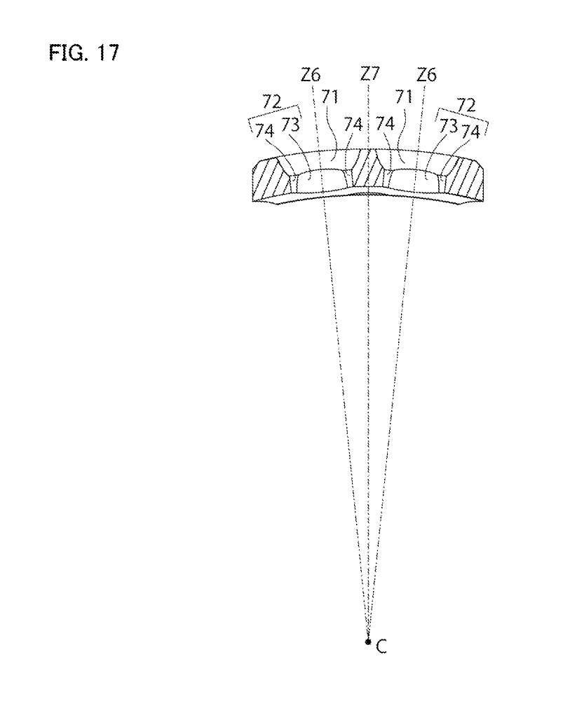

As illustrated in FIG. 3B, FIG. 10, and FIG. 17, each of the end portions 4 curves in an arc-like shape so that center axes Z6 of the two holes 7 intersect with each other at a center C of the arc. An angle of inclination (angle formed between an axis Z7 that connects a center point between the two holes 7 on the arc and the center C of the arc and the center axis Z6) of each of the holes 7 is set so that the axis of the screw 2 matches the center axis Z6 to prevent interference with the screw 2 driven similarly into the neighboring hole 7 when the screw 2 is driven into the hole 7.

The hole 7 does not have a region corresponding to the second regions 54 of the hole 5, but is otherwise formed similarly to the hole 5. The hole 7 includes an upper inner peripheral surface 71 on the upper surface side of the bone plate 1 and a lower inner peripheral surface 72 on the lower surface side of the bone plate 1. The upper inner peripheral surface 71 is formed along the shape of the upper portion of the head portion 21 of the screw 2. The upper inner peripheral surface 71 is formed of a conical surface so that an inner diameter thereof decreases toward the lower surface. The lower inner peripheral surface 72 includes a plurality of conical surfaces so that an inner diameter of the bone plate 1 in the longitudinal direction increases toward the lower surface as a whole. The lower inner peripheral surface 72 includes first regions 73 facing each other, each being inclined at a first angle (larger than 0 degrees; 15 degrees in FIG. 17) with respect to a center axis of the hole 7. The lower inner peripheral surface 72 includes second regions 74 facing each other in parallel to the center axis of the hole 7. One set of the first regions 73 facing each other across the center axis of the hole 7 therebetween is formed on the lower inner peripheral surface 72 so as to face each other in the longitudinal direction of the bone plate 1. Further, one set of the second regions 74 facing each other across the center axis of the hole 7 therebetween is formed on the lower inner peripheral surface 72 so as to face each other in the width direction of the bone plate 1.

The screw 2 can be driven into the hole 7 described above so that the axis of the screw 2 matches the center axis of the hole 7. Further, the screw 2 can be driven into the hole 7 so that the axis of the screw 2 is inclined in the longitudinal direction of the bone plate 1 with respect to the center axis of the hole 7.

One example of a method of using the above-mentioned bone plate system is now described. The holes 5 are described here, and the same applies to the holes 6 and the holes 7.

First, a surgeon places the lower surface of the bone plate 1 in contact with a target portion of a bone and determines an angle of the screw 2 with respect to the hole 5 in consideration of a surface shape of the bone, strength of the bone, and the like.

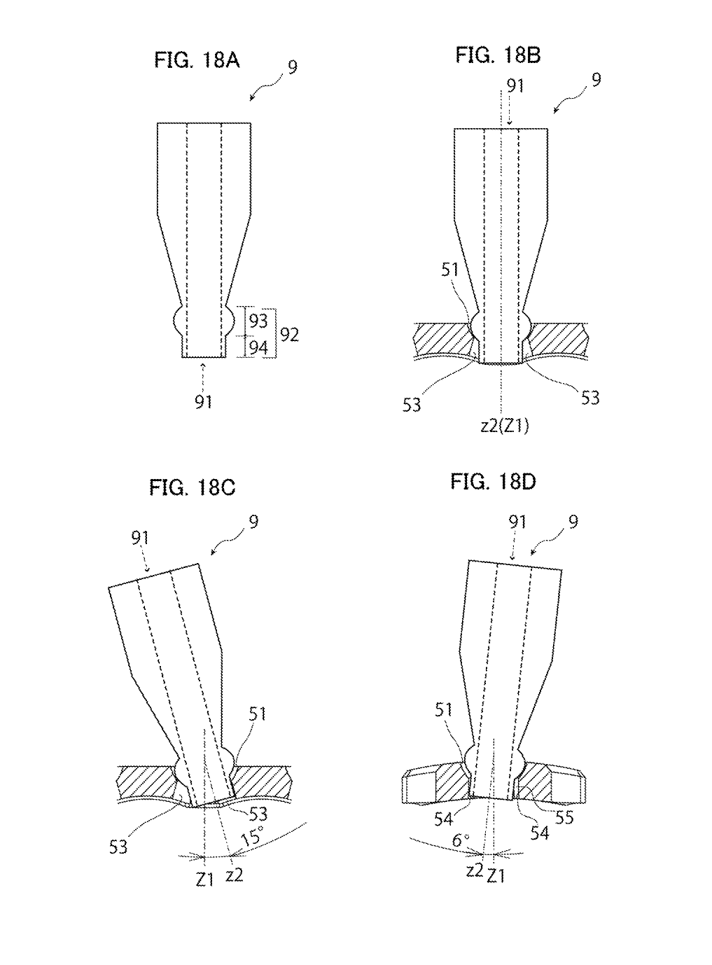

In this embodiment, the angle of the screw 2 is determined through use of a jig. FIG. 18A to FIG. 18D are side views for illustrating a schematic configuration of the jig. FIG. 18A is a side view for illustrating an internal configuration of the jig. FIG. 18B is a side view for illustrating a state in which the jig is placed upright. FIG. 18C is a side view for illustrating a state in which the jig is placed obliquely. FIG. 18D is another side view for illustrating a state in which the jig is placed obliquely. FIG. 18B and FIG. 18C are partial sectional views of the bone plate 1, taken along the longitudinal direction. FIG. 18D is a partial sectional view of the bone plate 1, taken along the width direction.

As illustrated in FIG. 18A, a jig 9 has an approximately cylindrical shape as a whole, and has a guide hole 91 configured to allow passage of a drill (for example, a drill having the same outer diameter as that of the shaft portion 22 of the screw 2) in a vertical direction therethrough. Further, the jig 9 has a distal end portion 92 to be brought into abutment against the hole 5. The distal end portion 92 includes an upper abutment portion 93 to be brought into abutment against the upper inner peripheral surface 51 of the hole 5 and a lower abutment portion 94 to be brought into abutment against the lower inner peripheral surface 52 of the hole 5. The upper abutment portion 93 is formed to have a spherical surface so as to perform a smooth rotational operation along the upper inner peripheral surface 51. An outer diameter (maximum outer diameter) of the upper abutment portion 93 is larger than the inner diameter (minimum inner diameter) of the hole 5. The lower abutment portion 94 is formed to have a cylindrical shape. An outer diameter of the lower abutment portion 94 is smaller than the inner diameter (minimum inner diameter) of the hole 5.

FIG. 18B is an illustration of a case where the jig 9 is placed into the hole 5 so that an axis z2 of the jig 9 matches the center axis Z1. FIG. 18C is an illustration of a side view for illustrating a case where the jig 9 is placed into the hole 5 so that the axis z2 of the jig 9 is inclined at the first angle (15 degrees in FIG. 18C) in the longitudinal direction of the bone plate 1 with respect to the center axis Z1. The angle of inclination of the jig 9 in the longitudinal direction of the bone plate 1 is limited up to the first angle by abutment of an outer peripheral surface of the lower abutment portion 94 against the first region 53 that is located on the side opposite to the direction of inclination. As a result, the angle of inclination of the guide hole 91 in the longitudinal direction is also limited up to the first angle. FIG. 18D is an illustration of a case where the jig 9 is placed into the hole 5 so that the axis z2 of the jig 9 is inclined at the second angle (6 degrees in FIG. 18D) in the width direction of the bone plate 1 with respect to the center axis Z1. The angle of inclination of the jig 9 in the width direction of the bone plate 1 is limited up to the second angle by abutment of the outer peripheral surface of the lower abutment portion 94 against the second region 54 that is located on the side opposite to the direction of inclination. As a result, the angle of inclination of the guide hole 91 in the width direction is also limited up to the second angle.

The surgeon determines the angle of inclination of the guide hole 91 (also an angle of the drill and an angle of the screw 2) through use of the jig 9. The surgeon can determine angles of the screw 2 to be fastened in the width direction and the longitudinal direction of the bone plate 1.

Next, the surgeon inserts the drill into the guide hole 91 of the jig 9 inclined at the determined angle to form in the bone a prepared hole for driving the shaft portion 22 of the screw 2 thereinto. A female thread may be formed on an inner surface of the prepared hole.

Finally, the surgeon inserts a distal end of the screw 2 into the prepared hole through the hole 5, and then tightens the screw 2 through use of a screw driver. The male thread 24 of the shaft portion 22 is engaged with the prepared hole. When the screw 2 is further tightened, the male thread 23 of the head portion 21 forms a female thread on the inner surface of the hole 5 so as to be engaged therewith.

In the above-mentioned manner, the screw 2 is fastened to the bone at an angle desired by the surgeon. Further, the screw 2 is fastened even to the bone plate 1 at an angle desired by the surgeon. The screw 2 is fastened to the bone at an appropriate angle in consideration of the surface shape of the bone, the strength of the bone, and the like, and hence the bone plate 1 is fixed to the bone and the screws 2.

The one embodiment of the present invention is described above. With the bone plate system of this embodiment, the screw can be fastened to the bone at a desired angle.

For example, the hole 5 of the bone plate 1 includes the first regions 53 and the second regions 54, each being inclined with respect to the center axis of the hole 5. As a result, the screw 2 can be fastened at a desired angle within a range of the angle of inclination of the first regions 53 and a range of the angle of inclination of the second regions 54. Further, for example, the female thread is not formed in advance on the inner surface of the hole 5 of the bone plate 1. As a result, the desired angle can be more easily determined.

Further, for example, the first regions 53 are arranged so as to face each other in the longitudinal direction of the bone plate 1, whereas the second regions 54 are arranged so as to face each other in the width direction of the bone plate 2. In general, the bone plate 1 is placed in contact with the bone so that the longitudinal direction of the bone plate 1 corresponds to a longitudinal direction of the bone. Therefore, the surgeon can simply set an axial direction of the screw 2 so that the axial direction of the screw 2 corresponds to the longitudinal direction of the bone or a width direction of the bone through use of the first regions 53 or the second regions 54.

The above-mentioned embodiment of the present invention is intended to exemplify the gist and the scope of the present invention without being limited thereto. For example, dimensions, shapes, and the like of the bone plate 1, the screws 2, and the jig 9 are not limited to the illustrated examples as long as the object of the present invention can be achieved. Further, for example, arrangements, numbers, dimensions, shapes, and the like of the holes 5, 6, and 7 are not limited to the illustrated examples as long as the object of the present invention can be achieved. Still further, a region and a range in which the head portion 21 of the screw 2 and the inner peripheral surface of each of the holes 5 to 7 are engaged with each other are not limited to the illustrated examples as long as the object of the present invention can be achieved. Still further, for example, the holes 5 may be arranged in place of the holes 6. Still further, for example, the holes 5 maybe arranged in place of the holes 7. Still further, for example, the holes 5 and the holes 7 do not strictly limit the inclinations of the screws 2 only in the longitudinal direction and the width direction, and may have play for allowing inclination in another direction. Still further, the holes 6 do not strictly limit the axial direction of the screw 2 to match the center axis of the hole 6, and may have play for allowing inclination in another direction.

The present invention can be carried out with various modifications within the gist of the present invention. Further, the above-mentioned embodiment and each of modified examples may be appropriately combined.

In the embodiment described above, the first angle (for example, 15 degrees) corresponding to the longitudinal direction of the bone plate 1 is set larger than the second angle (for example, 6 degrees) corresponding to the width direction of the bone plate 1. As another example of this configuration, for example, the lower inner peripheral surface 52 may be formed so that the first angle and the second angle are equal to each other. Further, for example, the lower inner peripheral surface 52 may be formed so that the first angle becomes smaller than the second angle. Still further, for example, the four angles (respective angles of the one set of first regions 53 and the one set of second regions 54) maybe freely set. Further, all the holes 5 are not required to have the same configuration. A configuration of at least one of the holes 5 may be changed. The first angle of the hole 7 may be set equal to the first angle or the second angle of the hole 5, or maybe set different from the first angle and the second angle of the hole 5. Further, all the holes 7 are not required to have the same configuration. A configuration of at least one of the holes 7 may be changed.

In the embodiment described above, the lower inner peripheral surface 52 is formed so that the screw 2 can be inclined in the longitudinal direction and the width direction of the bone plate 1. The direction of inclination is not limited to the longitudinal direction and the width direction of the bone plate 1. The lower inner peripheral surface 52 may be formed so that the screw 2 can be inclined in another direction (for example, a first direction intersecting with the longitudinal direction at 45 degrees and a second direction perpendicularly intersecting with the first direction). Further, all the holes 5 are not required to have the same configuration. A configuration of at least one of the holes 5 may be changed. Even for the holes 7, the lower inner peripheral surface 72 may be formed so that the screw 2 can be inclined in another direction different from the longitudinal direction. Still Further, all the holes 7 are not required to have the same configuration. A configuration of at least one of the holes 7 may be changed.

In the embodiment described above, the four inclined regions in total (the two first regions 53 and the two second regions 54) are formed on the lower inner peripheral surface 52 of the hole 5 so that the screw 2 can be inclined in the longitudinal direction and the width direction of the bone plate 1. The number of inclined regions may be from one to three, or may also be five or more. Further, in the embodiment described above, the two inclined regions in total (the two first regions 73) are formed on the lower inner peripheral surface 72 of the hole 7 so that the screw 2 can be inclined in the longitudinal direction of the bone plate 1. The number of inclined regions may be one, or may also be three or more.

The present invention can be provided in various forms, such as the bone plate system, the bone plate, and the method of fixing the bone plate. The present invention is applicable not only to small animals including dogs and cats but also to various creatures including human beings.

1. A bone plate, comprising:
an upper surface on a side opposite to a bone;
a lower surface on a side closer to the bone; and
at least one first hole connecting the upper surface and the lower surface and being configured to allow insertion of a screw,
the first hole having an upper inner peripheral surface on an upper surface side and a lower inner peripheral surface on a lower surface side, the upper inner peripheral surface having an inner diameter decreasing toward the lower surface, the lower inner peripheral surface having an inner diameter increasing toward the lower surface, the lower inner peripheral surface including at least one first region inclined at a first angle with respect to a center axis of the first hole and at least one second region inclined at a second angle with respect to the center axis of the first hole,
wherein the screw comprises a head portion and a shaft portion, each having a male thread formed thereon, and
wherein the lower inner peripheral surface has no female thread formed in advance, and the female thread is formed through rotation of the head portion after the screw is inserted into the first hole.

2. (canceled)

3. A bone plate according to claim 1, wherein the lower inner peripheral surface includes one set of the first regions facing each other across the center axis of the first hole and one set of the second regions facing each other across the center axis of the first hole.

4. A bone plate according to claim 1,
wherein the one set of first regions are arranged so as to face each other in a longitudinal direction of the bone plate, and
wherein the one set of second regions are arranged so as to face each other in a direction intersecting with the longitudinal direction of the bone plate.

5. A bone plate according to claim 1, wherein the first angle is larger than the second angle.

6. A bone plate according to claim 1,
wherein a plurality of the first holes are arranged at intervals in a longitudinal direction of the bone plate, and
wherein at least one of the plurality of first holes includes the one set of first regions arranged so as to face each other in the longitudinal direction of the bone plate and the one set of second regions arranged so as to face each other in the direction intersecting with the longitudinal direction of the bone plate.

7. A bone plate according to claim 1, further comprising at least one second hole connecting the upper surface and the lower surface and being configured to allow insertion of the screw,
the second hole having an upper inner peripheral surface on the upper surface side and a lower inner peripheral surface on the lower surface side, the upper inner peripheral surface having an inner diameter decreasing toward the lower surface, the lower inner peripheral surface having an inner diameter in a predetermined direction increasing toward the lower surface, the lower inner peripheral surface including at least one first region inclined at a first angle with respect to a center axis of the second hole and at least one second region in parallel to the center axis of the second hole.

8. A bone plate system, comprising:
the bone plate of claim 1; and
the screw of claim 1.
